(19) United States
(12) Patent Application Publication
(10) Pub. No.: US 2022/0204493 A1
(43) Pub. Date: Jun. 30, 2022

(54) CRYSTALLINE FORM OF A BET-INHIBITOR AND MANUFACTURE THEREOF

(71) Applicant: ORION CORPORATION, Espoo (FI)

(72) Inventors: Eila LUUKKONEN, Espoo (FI); Anna STAFFANS, Espoo (FI); Jan TOIS, Espoo (FI); Chandrasekhar ABBINENI, Hyderabad (IN); Roshaiah MARLA, Hyderabad (IN)

(21) Appl. No.: 17/602,594

(22) PCT Filed: Apr. 9, 2020
(86) PCT No.: PCT/FI2020/050235

§ 371 Date: Oct. 8, 2021

(30) Foreign Application Priority Data
Apr. 11, 2019 (FI) 20195292

(51) Int. Cl. C07D 413/14

(52) U.S. Cl. CPC C07D 413/14; C07B 2200/13

(57) ABSTRACT

The present invention relates to crystalline form 1 of 6-(3, 5-dimethylisoxazol-4-yl)-7-methoxy-3-methyl-1-(pyridin-2-ylmethyl)quinolin-2(1H)-one (I) and to a method of manufacture thereof. Compound (I) is a BET inhibitor useful in the treatment of cancer.

### TECHNICAL FIELD

The present invention relates to crystalline form 1 of 6-(3,5-dimethylisoxazol-4-yl)-7-methoxy-3-methyl-1-(pyridin-2-ylmethyl)quinolin-2 (1H)-one (I) and to method of manufacture thereof. Compound (I) is a BET inhibitor useful, for example, in the treatment of cancer.

### BACKGROUND OF THE INVENTION

The compound 6-(3,5-dimethylisoxazol-4-yl)-7-methoxy-3-methyl-1-(pyridin-2-ylmethyl)quinolin-2(1H)-one of formula (I) and derivatives thereof have been disclosed in WO 2015/104653. Compound of formula (I) is an inhibitor of Bromodomain and Extra-terminal motif (BET) proteins and has been found to be useful, for example, in the treatment of various cancers.

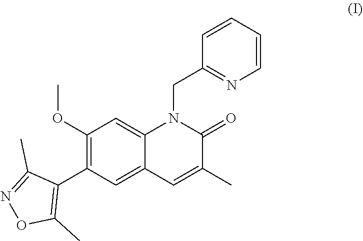 [SMILES: COc1cc2c(cc1-c1c(C)noc1C)cc(C)c(=O)n2Cc1ccccn1]

WO 2015/104653 discloses a process for the preparation of compound (I) via a Suzuki reaction starting from 6-bromo-7-methoxy-3-methyl-1-(pyridin-2-ylmethyl)quinolin-2(1H)-one intermediate of formula (Ia).

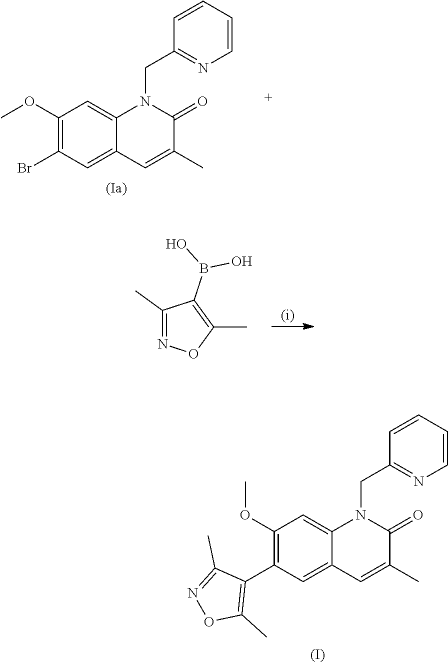

The process comprises dissolving intermediate (Ia) to a mixture of 1,2-dimethoxyethane (40 vol) and water (10 vol) followed by addition of (3,5-dimethylisoxazolyl)boronic acid (Ib), sodium carbonate and tetrakis triphenylphosphine palladium catalyst Pd(PPh₃)₄. After completion of reaction, the reaction mixture is diluted with large amount of EtOAc (500 vol), washed with large amounts of aqueous solutions (total 1000 vol), dried over sodium sulphate, filtered and concentrated. After these unit operations the residue is purified by preparative TLC to afford compound (I).

The above process suffers from several drawbacks. Firstly, the reaction is conducted in large volumes of an expensive and peroxide forming solvent. Secondly, high amount of expensive Pd-catalyst (5 mol-%) is needed. Thirdly, large volumes of solvents and aqueous solutions are used in the isolation process and large amount of organic solvents need to be distilled out during the concentration step making the method cumbersome for use in industrial scale. Finally, chromatographic purification of the end product is needed.

Thus, there is a need for a more practical and economical process that is suitable for large scale manufacture of compound (I) in crystalline form.

### SUMMARY OF THE INVENTION

It has now been found that the compound of formula (I) can be prepared using a process which is more practical and economical and suitable for a large scale production. In particular, the process enables easy purification of compound (I) by crystallization affording compound (I) in a stable crystalline form with high purity. The volumes of the solvents needed in the process are moderate. The amount of expensive reagents such as boronic acid derivative and palladium catalyst can be substantially reduced. The levels of palladium residues in the end product are also decreased. Moreover, it was found that the crystalline polymorphic form 1 which is obtained as the end product is physically stable, has low hygroscopicity, can be obtained in consistent manner, is not in the form of a solvate and is easy to mill and filter making it particularly suitable as a pharmaceutical ingredient for use in the manufacture of stable pharmaceutical dosage forms.

Thus, in one aspect, the present invention provides 6-(3,5-dimethylisoxazol-4-yl)-7-methoxy-3-methyl-1-(pyridin-2-ylmethyl)quinolin-2(1H)-one (I) in crystalline form 1.

In another aspect, the present invention provides a method for preparing 6-(3,5-dimethyl-isoxazol-4-yl)-7-methoxy-3-methyl-1-(pyridin-2-ylmethyl)quinolin-2(1H)-one (I) crystalline form I, comprising the steps of

a) reacting 6-bromo-7-methoxy-3-methyl-1-(pyridin-2-ylmethyl)quinolin-2(1H)-one of formula (Ia)

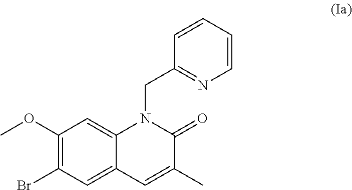 [SMILES: COc1cc2c(cc1Br)cc(C)c(=O)n2Cc1ccccn1]

with a boronic acid derivative of formula (Ib) or (Ic)

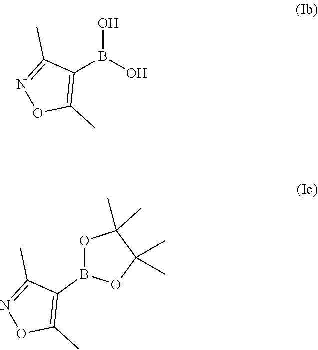

at an elevated temperature in the presence of a palladium catalyst and a base in an acetonitrile-water or n-butanol-water solvent,

b) optionally isolating the organic phase of the reaction mixture;

c) adding toluene and optionally water to the reaction mixture or to the organic phase of the reaction mixture if it was isolated in the previous step;

d) isolating the organic phase if water was added in the previous step;

e) concentrating the organic phase by distillation; and

f) cooling the concentrated organic phase and isolating the precipitated compound (I).

In another aspect, the present invention provides a method for preparing 6-(3,5-dimethyl-isoxazol-4-yl)-7-methoxy-3-methyl-1-(pyridin-2-ylmethyl)quinolin-2(1H)-one (I) crystalline form 1, comprising the steps of

a) reacting 6-bromo-7-methoxy-3-methyl-1-(pyridin-2-ylmethyl)quinolin-2(1H)-one of formula (Ia)

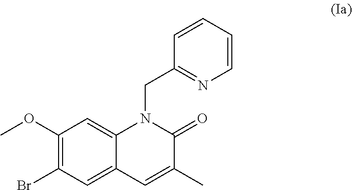 [SMILES: COc1cc2c(cc1Br)cc(C)c(=O)n2Cc1ccccn1]

with a boronic acid derivative of formula (Ib)

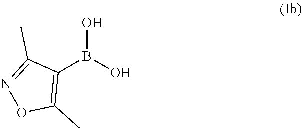

at an elevated temperature in the presence of a palladium catalyst and a base in an acetonitrile-water solvent,

b) optionally concentrating the reaction mixture by distillation;

c) adding toluene and water to the reaction mixture;

d) isolating the organic phase;

e) concentrating the organic phase by distillation; and

f) cooling the concentrated organic phase and isolating the precipitated compound (I).

In another aspect, the present invention provides a method for preparing 6-(3,5-dimethyl-isoxazol-4-yl)-7-methoxy-3-methyl-1-(pyridin-2-ylmethyl)quinolin-2(1H)-one (I) crystalline form 1, comprising the steps of

a) reacting 6-bromo-7-methoxy-3-methyl-1-(pyridin-2-ylmethyl)quinolin-2(1H)-one of formula (Ia)

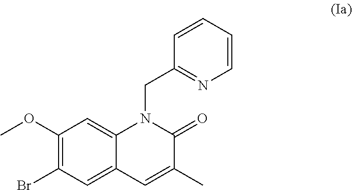 [SMILES: COc1cc2c(cc1Br)cc(C)c(=O)n2Cc1ccccn1]

with a boronic acid derivative of formula (Ic)

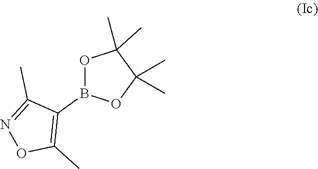 [SMILES: Cc1noc(C)c1B1OC(C)(C)C(C)(C)O1]

at an elevated temperature in the presence of a palladium catalyst and a base in a n-butanol-water solvent,

b) optionally concentrating the reaction mixture by distillation;

c) adding toluene and water to the reaction mixture;

d) isolating the organic phase;

e) concentrating the organic phase by distillation; and

f) cooling the concentrated organic phase and isolating the precipitated compound (I).

In still another aspect, the present invention provides method of preparing 6-(3, 5-dimethylisoxazol-4-yl)-7-methoxy-3-methyl-1-(pyridin-2-ylmethyl)quinolin-2 (1H)-one (I) crystalline form 1, comprising the steps of

a) reacting 6-bromo-7-methoxy-3-methyl-1-(pyridin-2-ylmethyl)quinolin-2(1H)-one of formula (Ia)

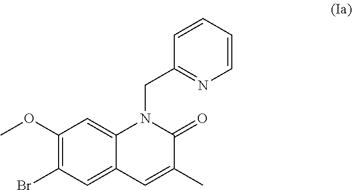 [SMILES: COc1cc2c(cc1Br)cc(C)c(=O)n2Cc1ccccn1]

with a boronic acid derivative of formula (Ic)

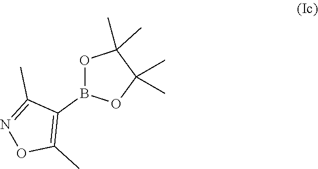 [SMILES: Cc1noc(C)c1B1OC(C)(C)C(C)(C)O1]

at an elevated temperature in the presence of a palladium catalyst and a base in a n-butanol-water solvent,

b) isolating the organic phase of the reaction mixture;

c) adding toluene to the organic phase;

d) concentrating the organic phase by distillation; and

f) cooling the concentrated organic phase and isolating the precipitated compound of formula (I).

### BRIEF DESCRIPTION OF THE DRAWINGS

FIG. 1 shows the X-ray powder diffraction pattern of crystalline form 1 of compound (I) from an unmilled sample.

FIG. 2 shows the X-ray powder diffraction pattern of crystalline form 1 of compound (I) from a milled sample.

FIG. 3 shows the X-ray powder diffraction pattern of crystalline form 1 of compound (I) from an unmilled sample and a milled sample.

FIG. 4 shows the X-ray powder diffraction pattern of amorphic form of compound (I).

FIG. 5 shows the X-ray powder diffraction pattern of crystalline form 2 of compound (I) from a milled sample.

### DETAILED DESCRIPTION OF THE INVENTION

The term “mol-% of palladium catalyst”, as used herein, refers to the percentage of the amount of palladium catalyst (in moles) used in the reaction step in relation to the amount of starting compound (in moles). For example, if 0.01 mol of palladium catalyst, for example Pd(PPh₃)₄, is used per 1 mol of compound (Ia) in the reaction step a), the mol-% of palladium catalyst used in step a) is (0.01/1)*100 mol-%=1 mol-%.

The present invention provides 6-(3,5-dimethylisoxazol-4-yl)-7-methoxy-3-methyl-1-(pyridin-2-ylmethyl)quinolin-2(1H)-one (I) in crystalline form 1.

Crystalline form 1 of compound (I) has been characterized by X-ray powder diffraction (XRPD) studies.

Accordingly, in one aspect, the present invention provides crystalline form 1 of compound (I) having a X-ray powder diffraction pattern comprising characteristic peaks at about 10.8, 25.6 and 30.7 degrees 2-theta.

In another aspect, the present invention provides crystalline form 1 of compound (I) having a X-ray powder diffraction pattern comprising characteristic peaks at about 10.8, 14.7, 25.6 and 30.7 degrees 2-theta.

In still another aspect, the present invention provides crystalline form 1 of compound (I) having a X-ray powder diffraction pattern comprising characteristic peaks at about 8.3, 10.8, 11.8, 14.7, 18.2, 25.6 and 30.7 degrees 2-theta.

In still another aspect, the present invention provides crystalline form 1 of compound (I) having a X-ray powder diffraction pattern comprising characteristic peaks at about 8.3, 9.1, 10.8, 11.8, 14.7, 18.2, 25.6 and 30.7 degrees 2-theta.

In still another aspect, the present invention provides crystalline form 1 of compound (I) having a X-ray powder diffraction pattern comprising characteristic peaks at 8.3, 9.1, 10.8, 11.8, 14.7, 18.2, 20.6, 22.4, 23.2, 25.6, 28.6 and 30.7 degrees 2-theta.

The above characteristics peaks refer to X-ray powder diffraction pattern measured from a milled sample.

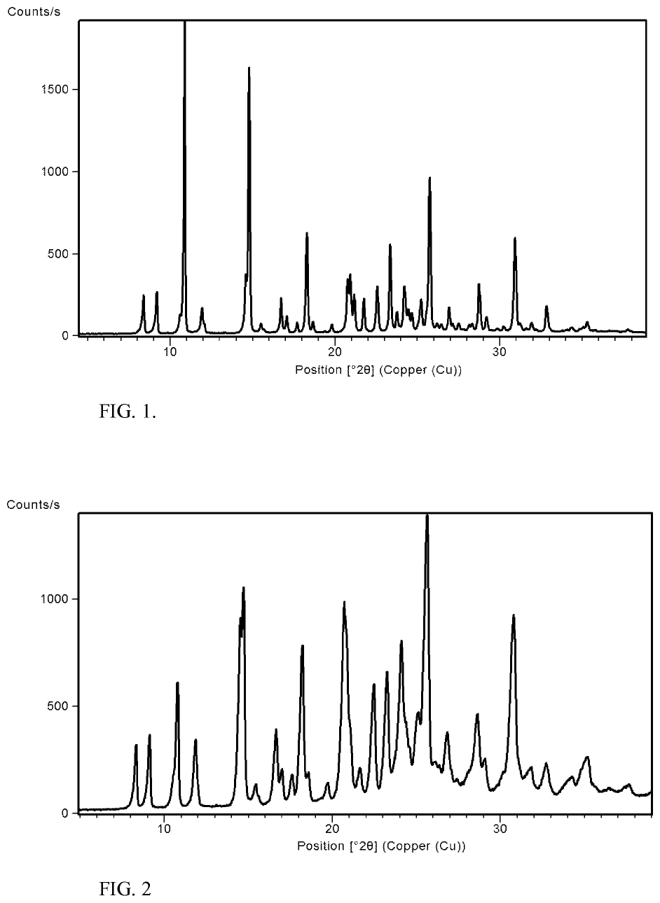

In yet a further aspect, the crystalline form 1 of compound (I) is further characterized by an X-ray powder diffraction pattern as depicted in FIG. 1 or 2.

In yet a further aspect, the crystalline form 1 of compound (I) is an anhydrate.

In yet another aspect, the present invention provides 6-(3,5-dimethylisoxazol-4-yl)-7-methoxy-3-methyl-1-(pyridin-2-ylmethyl)quinolin-2(1H)-one (I) in crystalline form 1 as defined herein, substantially free of any other crystalline form of compound (I).

In yet another aspect, the present invention provides 6-(3,5-dimethylisoxazol-4-yl)-7-methoxy-3-methyl-1-(pyridin-2-ylmethyl)quinolin-2(1H)-one (I) in crystalline form 1 having chemical purity of at least 98 w-%, preferably at least 99 w-%, more preferably at least 99.5 w-%, for example at least 99.8 w-%.

It is recognized by the skilled person that the X-ray powder diffraction pattern peak positions referred to herein can be subject to variations of +/−0.2 degrees 2-theta according to various factors such as temperature, concentration, sample handling and instrumentation used. Therefore, signals and peak positions are referred to herein as being at “about” specific values.

In accordance with the present invention 6-(3,5-dimethylisoxazol-4-yl)-7-methoxy-3-methyl-1-(pyridin-2-ylmethyl)quinolin-2(1H)-one (I) crystalline form 1 is prepared by

a) reacting 6-bromo-7-methoxy-3-methyl-1-(pyridin-2-ylmethyl)quinolin-2(1H)-one of formula (Ia)

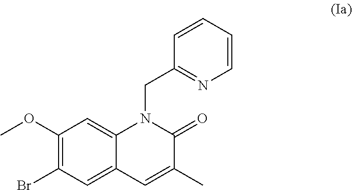 [SMILES: COc1cc2c(cc1Br)cc(C)c(=O)n2Cc1ccccn1]

with a boronic acid derivative of formula (Ib) or (Ic)

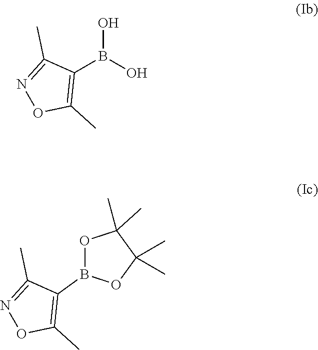

at an elevated temperature in the presence of a palladium catalyst and a base in an acetonitrile-water or n-butanol-water solvent,

b) optionally isolating the organic phase of the reaction mixture;

c) adding toluene and optionally water to the reaction mixture or to the organic phase of the reaction mixture if it was isolated in the previous step;

d) isolating the organic phase if water was added in the previous step;

e) concentrating the organic phase by distillation; and

f) cooling the concentrated organic phase and isolating the precipitated compound (I).

In accordance with one embodiment the present invention, 6-(3,5-dimethylisoxazol-4-yl)-7-methoxy-3-methyl-1-(pyridin-2-ylmethyl)quinolin-2(1H)-one (I) crystalline form 1 can be prepared by

a) reacting 6-bromo-7-methoxy-3-methyl-1-(pyridin-2-ylmethyl)quinolin-2(1H)-one of formula (Ia)

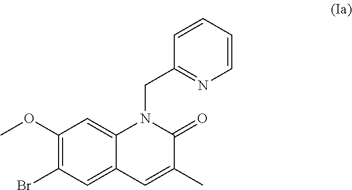 [SMILES: COc1cc2c(cc1Br)cc(C)c(=O)n2Cc1ccccn1]

with a boronic acid derivative of formula (Ib)

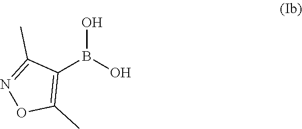

at an elevated temperature in the presence of a palladium catalyst and a base in an acetonitrile-water solvent,

b) optionally concentrating the reaction mixture by distillation;

c) adding toluene and water to the reaction mixture;

d) isolating the organic phase;

e) concentrating the organic phase by distillation; and

f) cooling the concentrated organic phase and isolating the precipitated compound (I).

For carrying out the Suzuki reaction with the boronic acid derivative of formula (Ib), the mixture of acetonitrile, water, the base, the palladium catalyst and 6-bromo-7-methoxy-3-methyl-1-(pyridin-2-ylmethyl)quinolin-2(1H)-one of formula (Ia) is suitably charged to a reactor vessel under nitrogen atmosphere. In the acetonitrile-water solvent, the ratio of acetonitrile to water is generally from about 40:60 to about 90:10, preferably from about 50:50 to about 85:15, more preferably from about 60:40 to about 80:20, for example 75:25, by volume. The base is suitably an inorganic base, e.g. inorganic carbonate or bicarbonate, such as potassium carbonate or sodium carbonate. Potassium carbonate is preferred. Palladium catalyst is preferably a soluble palladium catalyst such as tetrakis triphenylphosphine palladium catalyst Pd(PPh₃)₄ or a combination of Pd(OAc)₂ and triphenylphosphine wherein the molar of the Pd(OAc)₂ to triphenylphosphine is suitably about 1:3. Pd(PPh₃)₄ is particularly preferred. The amount of palladium catalyst used per amount of compound of formula (Ia) in step a) is from about 0.3 to about 2 mol-%, preferably from about 0.5 to about 1.5 mol-%, more preferably from about 0.6 to about 1.2 mol-%. The amount of acetonitrile-water solvent to be used is suitably 2-5 kg, for example 3-4 kg, per 1 kg of 6-bromo-7-methoxy-3-methyl-1-(pyridin-2-ylmethyl)quinolin-2(1H)-one of formula (Ia).

The reaction mixture is heated to a temperature from about 60 to about 80° C., preferably at 70±3° C. The (3,5-dimethylisoxazolyl)boronic acid (Ib) is preferably dissolved into a mixture of acetonitrile and water in a separate vessel under nitrogen atmosphere and then added slowly to the hot reaction mixture. This reduces the possibility of degradation of boronic acid compound during the heating of the reaction mixture. The boronic acid compound (Ib) is suitably used in an amount of 1 to 2 molar equivalents, for example about 1.5 molar equivalents, per one molar equivalent of starting material (Ia). The reaction mixture is then refluxed for a time period sufficient to complete the reaction, typically from about 2 to about 16 h, for example 6-8 h.

If desired, after completing the reaction, the reaction mixture may be concentrated by distillation. Typically, at least about 50 w-%, more typically at least about 60 w-%, for example about 60-90 w-%, of the solvent can be distilled off from the reaction mixture. However, it is also possible to proceed to the next step without concentrating the reaction mixture.

In the next step, toluene and water are added to the stirred reaction mixture under heating. The amount of toluene-water to be added is suitably such that after the addition there is about 8-12 kg, for example 9-10 kg, of solvent per 1 kg of starting material 6-bromo-7-methoxy-3-methyl-1-(pyridin-2-ylmethyl)quinolin-2(1H)-one of formula (Ia). The ratio of toluene to water is suitably from about 40:60 to about 80:20, typically from about 45:55 to about 75:25, preferably from about 50:50 to about 70:30, more preferably from about 55:45 to about 65:35, for example 60:40, by volume. Thereafter, the phases can be separated while hot and the organic phase is suitably filtered, for example at about 70-80° C., through celite (diatomaceous earth). It was found that the celite filtration was effective to remove most of the soluble palladium catalyst from the reaction mixture.

The filtrate (organic phase) is then suitably concentrated by distilling. Generally, at least about 50 w-%, typically at least about 60 w-%, more typically about 60-90 w-%, for example 70-80 w-%, of the solvent may be distilled off from the filtrate. At the end of the distillation, the amount of hot solvent is suitably about 1.5-5 kg, for example about 1.6-3 kg, per 1 kg of the end product. During the distillation of the organic phase, also acetonitrile and water are removed from the organic (toluene) phase which increases the yield and ascertains that pure crystalline form 1 is obtained during the subsequent crystallization step.

The compound of formula (I) can then be precipitated as crystalline form 1 by cooling the concentrated mixture slowly to lower than 20° C., preferably to lower than 10° C., such as from 0 to 10° C., for example to about 5° C., and stirred for a period sufficient to complete the precipitation of the compound of formula (I), for example for about 6 to 24 h. The precipitated product can be isolated, for example by filtering, and washed with water and isopropanol, and dried, for example, at reduced pressure.

If desired, the precipitated compound of formula (I) can be recrystallized, for example, by dissolving the product into isopropanol with heating, for example to about 80° C., followed by filtration. The amount of isopropanol used is suitably about 5-15 kg, preferably about 6-10 kg, per 1 kg of the end product. If desired, the filtrate can be concentrated before the crystallization by distillation. Generally, more than about 20 w-%, typically more than about 25 w-%, more typically about 30-60 w-%, for example about 50 w-%, of the isopropanol may be distilled off. At the end of the distillation, the amount of isopropanol solvent is suitably about 2-10 kg, for example about 3-6 kg, per 1 kg of the end product. The concentrated isopropanol mixture can then be cooled slowly, for example at the rate of 5-10° C./h, to lower than 20° C., preferably to lower than 10° C., such as from 0 to 10° C., for example to about 5° C., and stirred for a period sufficient to complete the precipitation of the compound of formula (I), for example for about 1 to 6 h. The precipitated product can be isolated, for example by filtering, and washed with water and isopropanol, and dried, for example, at reduced pressure to afford compound of formula (I) as crystalline form 1.

In accordance with one embodiment the present invention, 6-(3,5-dimethylisoxazol-4-yl)-7-methoxy-3-methyl-1-(pyridin-2-ylmethyl)quinolin-2(1H)-one (I) crystalline form 1 can be prepared by

a) reacting 6-bromo-7-methoxy-3-methyl-1-(pyridin-2-ylmethyl)quinolin-2(1H)-one of formula (Ia)

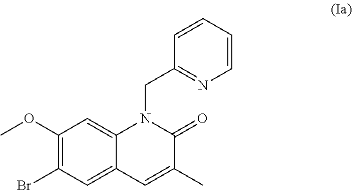 [SMILES: COc1cc2c(cc1Br)cc(C)c(=O)n2Cc1ccccn1]

with a boronic acid derivative of formula (Ic)

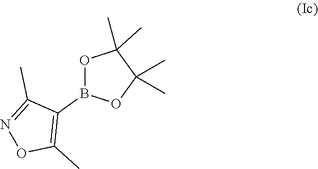 [SMILES: Cc1noc(C)c1B1OC(C)(C)C(C)(C)O1]

at an elevated temperature in the presence of a palladium catalyst and a base in a n-butanol-water solvent,

b) optionally concentrating the reaction mixture by distillation;

c) adding toluene and water to the reaction mixture;

d) isolating the organic phase;

e) concentrating the organic phase by distillation; and

f) cooling the concentrated organic phase and isolating the precipitated compound (I).

In accordance with another embodiment the present invention, 6-(3,5-dimethylisoxazol-4-yl)-7-methoxy-3-methyl-1-(pyridin-2-ylmethyl)quinolin-2(1H)-one (I) crystalline form 1 can be prepared by

a) reacting 6-bromo-7-methoxy-3-methyl-1-(pyridin-2-ylmethyl)quinolin-2 (1H)-one of formula (Ia)

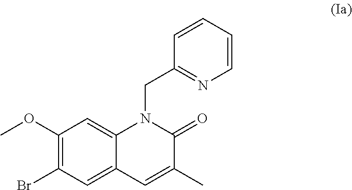 [SMILES: COc1cc2c(cc1Br)cc(C)c(=O)n2Cc1ccccn1]

with a boronic acid derivative of formula (Ic)

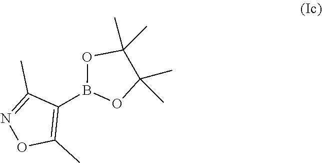 [SMILES: Cc1noc(C)c1B1OC(C)(C)C(C)(C)O1]

at an elevated temperature in the presence of a palladium catalyst and a base in a n-butanol-water solvent,

b) isolating the organic phase of the reaction mixture;

c) adding toluene to the organic phase;

d) concentrating the organic phase by distillation; and

e) cooling the concentrated organic phase and isolating the precipitated compound of formula (I).

For carrying out the Suzuki reaction with the boronic acid derivative of formula (Ic), the mixture of n-butanol, water, the base, the palladium catalyst, 3,5-dimethyl-4-(4,4,5,5-tetramethyl-1,3,2-dioxaborolan-2-yl)isoxazole (Ic) and 6-bromo-7-methoxy-3-methyl-1-(pyridin-2-ylmethyl)-quinolin-2(1H)-one of formula (Ia) is suitably charged to a reactor vessel under nitrogen atmosphere. In the n-butanol-water solvent, the ratio of n-butanol to water is generally from about 50:50 to about 90:10, more preferably from about 70:30 to about 85:15, for example about 80:20, by volume. The base is suitably an inorganic base, e.g. inorganic carbonate or bicarbonate, such as potassium carbonate or sodium carbonate. Potassium carbonate is preferred. Palladium catalyst is preferably a soluble palladium catalyst such as tetrakis triphenylphosphine palladium catalyst Pd(PPh₃)₄ or a combination of Pd(OAc)₂ and triphenylphosphine wherein the molar of the Pd(OAc)₂ to triphenylphosphine is suitably about 1:3. The amount of palladium catalyst used per amount of compound of formula (Ia) in step a) is from about 0.3 to about 2 mol-%, preferably from about 0.5 to about 1.5 mol-%, more preferably from about 0.6 to about 1.2 mol-%. The amount of n-butanol-water solvent to be used is suitably 2-6 kg, for example 3-5 kg, per 1 kg of 6-bromo-7-methoxy-3-methyl-1-(pyridin-2-ylmethyl)quinolin-2(1H)-one of formula (Ia).

The reaction mixture is heated to a temperature from about 60 to about 100° C., for example to refluxing temperature. The reaction mixture can then be refluxed for a time period sufficient to complete the reaction, typically from about 2 to about 16 h, for example 3-6 h.

If desired, after completing the reaction, the water phase can be separated off from the hot reaction mixture followed by proceeding to the next step with the isolated organic phase. However, it is also possible to proceed with the reaction mixture as such without isolation of the organic phase.

If desired, after completing the reaction, the reaction mixture or the isolated organic phase may be concentrated by distillation. Typically, at least about 50 w-%, more typically at least about 60 w-%, for example about 60-90 w-%, of the solvent can be distilled off from the reaction mixture. However, it is also possible to proceed to the next step without concentrating the reaction mixture.

In the next step, toluene and optionally water are added to the stirred reaction mixture or to the isolated organic phase under heating. The amount of toluene or toluene-water to be added is suitably such that after the addition there is about 5-10 kg, for example 6-8 kg, of solvent per 1 kg of starting material 6-bromo-7-methoxy-3-methyl-1-(pyridin-2-ylmethyl)quinolin-2(1H)-one of formula (Ia). If the combination of toluene and water is used, the ratio of toluene to water is suitably from about 40:60 to about 80:20, typically from about 45:55 to about 75:25, preferably from about 50:50 to about 70:30, more preferably from about 55:45 to about 65:35, for example 60:40, by volume.

Thereafter, the phases can be separated while hot and the organic phase is suitably filtered, for example at about 70-80° C., through celite (diatomaceous earth). It was found that the celite filtration was effective to remove most of the soluble palladium catalyst from the reaction mixture.

The obtained filtrate is then suitably concentrated by distilling. Generally, at least about 50 w-%, typically at least about 60 w-%, more typically about 60-90 w-%, for example 70-80 w-%, of the solvent may be distilled off from the filtrate. At the end of the distillation, the amount of solvent is suitably about 1.5-5 kg, for example about 1.6-3 kg, per 1 kg of the end product.

The compound of formula (I) can then be precipitated as crystalline form 1 by cooling the concentrated mixture slowly to lower than 20° C., preferably to lower than 10° C., such as from 0 to 10° C., for example to about 5° C., and stirred for a period sufficient to complete the precipitation of the compound of formula (I), for example for about 6 to 24 h. The precipitated product can be isolated, for example by filtering, and washed with water and isopropanol, and dried, for example, at reduced pressure.

If desired, the precipitated compound of formula (I) can be recrystallized, for example, by dissolving the product into isopropanol with heating followed by filtration. The amount of isopropanol used is suitably about 5-15 kg, preferably about 6-10 kg, per 1 kg of the end product. If desired, the filtrate can be concentrated before the crystallization by distillation. Generally, more than about 20 w-%, typically more than about 25 w-%, more typically about 30-60 w-%, for example about 50 w-%, of the isopropanol may be distilled off. At the end of the distillation, the amount of isopropanol solvent is suitably about 3-10 kg, for example about 4-7 kg, per 1 kg of the end product. At the end of the distillation, the amount of isopropanol solvent is suitably about 2-10 kg, for example about 3-6 kg, per 1 kg of the end product. The concentrated isopropanol mixture can then be cooled slowly, for example at the rate of 5-10° C./h, to lower than 20° C., preferably to lower than 10° C., for example to about 5° C., and stirred for a period sufficient to complete the precipitation of the compound of formula (I), for example for about 1 to 6 h. The precipitated product can be isolated, for example by filtering, and washed with water and isopropanol, and dried, for example, at reduced pressure to afford compound of formula (I) as crystalline form 1.

The compound of formula (Ia) can be prepared according to methods disclosed in WO 2015/104653.

Alternatively, and preferably, the compound of formula (Ia) is prepared by a method comprising the steps of

(i) reacting 4-bromo-methoxyaniline with propionic anhydride in a solvent to obtain a compound of formula (IV);

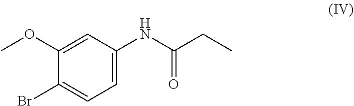

(ii) treating the compound of formula (IV) with phosphorous oxychloride and dimethyl formamide to obtain a compound of formula (III);

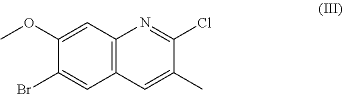

(iii) treating the compound of formula (III) with acetic acid and water at elevated temperature to obtain a compound of formula (II); and

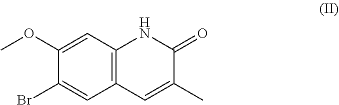

(iv) reacting the compound of formula (II) with 2-(chloromethyl)pyridine or a salt thereof in a solvent at elevated temperature in the presence of a base followed by isolation of the obtained compound of formula (Ia).

Step (i) is suitably carried out in acetonitrile-water solvent. The ratio of acetonitrile to water is generally from about 10:90 to about 30:70, for example about 20:80, by volume. The amount of acetonitrile-water solvent to be used is suitably 3-8 kg, for example 4-6 kg, per 1 kg of 4-bromo-methoxyaniline. The amount of propionic anhydride to be used is suitably about 1-2 mol equivalents to one mol equivalent of 4-bromo-methoxyaniline. The reaction is suitably carried out at elevated temperature, for example at about 50-70° C. for a time sufficient to complete the reaction, typically about 1-2 hours. Thereafter water is added to the reaction mixture and the precipitated compound of formula (IV) is isolated for example by filtrating, washed, for example, with water and dried under reduced pressure.

Step (ii) is suitably carried out neat (without any further solvent) or, if solvent is used, in toluene solvent. The amount of toluene solvent is suitably 0.3-2 kg, for example 0.5-1 kg, per 1 kg of compound of formula (IV). The amount of dimethyl formamide and phosphorous oxychloride to be used is suitably about 1-2 molar equivalents and about 3-4 molar equivalents, respectively, to one molar equivalent of compound of formula (IV). The reaction is suitably carried out first at about 20° C. to about 30° C., followed by heating to about 60-90° C. under stirring for a time sufficient to complete the reaction, for example about 1-2 hours. Thereafter, the reaction mixture is cooled to about room temperature, and water and 50% sodium hydroxide solution is added. The obtained compound of formula (III) can be extracted, for example, to toluene suitably at elevated temperature, for example at about 70-90° C. The toluene extract can be concentrated by distilling off part of toluene. The residue can be used in the next step without isolation of the compound of formula (III).

In step (iii) a solution of acetic acid to water is added to the extraction residue from the previous step. The ratio of acetic acid to water is generally from about 90:10 to about 99:1, for example about 98:2, by weight. The reaction is suitably carried out by refluxing the reaction mixture for a time sufficient to complete the reaction, typically about 10-30 hours, for example 12 hours. If desired, the reaction mixture can then be concentrated by distilling. More than about 25 w-%, typically more than about 30 w-%, for example about 35-60 w-%, of the solvent may be distilled off. Thereafter water is added slowly to the reaction mixture at the temperature of, for example, about 90° C., followed by stirring for 1-2 hours and cooling, for example to about room temperature. The precipitated compound (II) can be isolated for example by filtrating, washed, for example, with water and dried under reduced pressure.

In step (iv) the solvent is preferably toluene-water. The ratio of toluene to water is generally from about 50:50 to about 95:5, more preferably from about 75:25 to about 90:10, for example about 85:15, by weight. The base is suitably potassium hydroxide. A phase transfer catalyst such as tetrabutylammonium bromide is suitably also used. The amount of toluene-water solvent to be used is suitably 4-10 kg, for example 6-8 kg, per 1 kg of compound of formula (II). The reaction is generally carried out at the temperature from about 50° C. to 100° C., for example at about 80° C., for a time sufficient to complete the reaction, typically about 1-2 hours. The phases can be separated hot and some more toluene can be added to the organic phase. If desired, the organic phase can be then concentrated by distillation. The residue can next be cooled to lower than about 20° C., such as from 0 to 15° C., for example to about 10° C. The crystallized compound of formula (Ia) can be isolated for example by filtrating, washed for example with water and acetonitrile and dried under reduced pressure.

The crystalline form I of compound (I) can be formulated into pharmaceutical dosage forms such as tablets, capsules, powders or suspensions together with one or more excipients which are known in the art.

The invention is further illustrated by the following non-limiting examples.

### Example 1. Preparation of N-(4-Bromo-3-methoxyphenyl)propionamide (IV)

Acetonitrile (18 kg), 4-bromo-methoxyaniline (22.5 kg) and water (90 kg) were charged to a reactor and the mixture was heated to about 60° C. Propionic anhydride (21.7 kg) was added slowly. The reaction mixture was stirred at the reaction temperature for about one hour. When the reaction was complete, water was added resulting the solid precipitation. The crystalline compound was collected at 20° C., washed with water and finally dried under reduced pressure to afford the title compound (yield 26.6 kg/92.7%, purity 99.9%).

### Example 2. Preparation of 6-Bromo-2-chloro-7-methoxy-3-methylquinoline (III)

Dimethyl formamide (10.2 kg), toluene (9.7 kg) and N-(4-bromo-3-methoxy-phenyl)propionamide (26 kg) were charged to a reactor. The formed solution was added slowly to another reactor containing phosphorous oxychloride (53.1 kg) and toluene (11.3 kg) while maintaining the temperature between 20 and 30° C. The reaction mixture was then agitated for an additional hour at about 30° C. The mixture was heated to the reaction temperature about 80° C. and stirred for about one hour. After that, the mixture was cooled to 25° C. Half of the reaction mixture was transferred to a reactor containing water (130 kg) while maintaining the temperature about 30° C. Next 50% sodium hydroxide solution (55 kg) was added followed by the rest of the reaction mixture. Finally, 6-bromo-2-chloro-7-methoxy-3-methylquinoline was extracted to toluene (100 kg) at about 80° C. and concentrated by distilling off part of toluene. The residue was used in the next step.

### Example 3. Preparation of 6-Bromo-7-methoxy-3-methylquinolin-2(1H)-one (II)

Acetic acid (218.4 kg) and water (3.6 kg) were added to the solution from Example 2. The reaction mixture was refluxed until the reaction was complete, for about 12 hours. About 140 l of the solvents were distilled off. The reactor content was cooled to about 90° C. and water (52 kg) was added slowly. Stirring was continued at 90° C. for about an hour. The mixture was cooled to about 20° C. The solid was collected by filtration, washed with water and dried under reduced pressure to yield 6-bromo-7-methoxy-3-methylquinolin-2(1H)-one (19.7 kg/73.1%, purity 100%).

### Example 4. Preparation of 6-Bromo-7-methoxy-3-methyl-1-(pyridin-2-ylmethyl)quinolin-2(1H)-one (Ia)

Water (19.1 kg), 2-(chloromethyl)pyridine hydrochloride (12.2 kg), 6-bromo-7-methoxy-3-methylquinolin-2(1H)-one (15.4 kg), toluene (89.1 kg) and tetrabutylammonium bromide (1.87 kg) were charged to a reactor. The reactor content was heated to 80° C. and 46% potassium hydroxide (28.0 kg) solution was added slowly. The reaction mixture was refluxed until the reaction no longer proceeded. The phases were separated at 80° C. Toluene (13.4 kg) was added to the organic phase. The mixture was concentrated by distillation. The residue was cooled to 10° C. 6-Bromo-7-methoxy-3-methyl-1-(pyridin-2-ylmethyl)quinolin-2(1H)-one was collected by filtration, washed with water and acetonitrile to yield 15.0 kg/72.5%, purity 98.6%.

### Example 5. Preparation of 6-(3, 5-Dimethylisoxazol-4-yl)-7-methoxy-3-methyl-1-(pyridin-2-ylmethyl)quinolin-2(1H)-one (I) crystalline form 1

Acetonitrile (50 kg), water (21.0 kg), 6-bromo-7-methoxy-3-methyl-1-(pyridin-2-ylmethyl)quinolin-2(1H)-one (21.0 kg), potassium carbonate (24.2 kg) and tetrakis(triphenylphosphine)palladium (0.63 kg) were charged to a reactor. The mixture was heated about to 70° C. In a separate reactor, (3,5-dimethylisoxazol-4-yl)boronic acid (12.40 kg) was dissolved into acetonitrile (41.3 kg) and water (13.7 kg). Next (3,5-dimethylisoxazol-4-yl)boronic acid solution was added to the first reactor at 65-70° C. The reaction mixture was refluxed for about 8 hours. When the reaction was complete, the reaction mixture was concentrated by distilling off about 75 kg of the solvents. To the residue, toluene (87.4 kg) and water (63 kg) were added. The phases were separated at about 70° C. The hot organic phase was filtered through celite. Hot toluene (33.7 kg) was used to flush the filter. Combined filtrates were concentrated by distilling off about 91 kg of the solvents. The residue was cooled and the solid was collected by filtration at about 5° C., washed with water and isopropanol and finally dried under reduced pressure to yield the crude title compound (18.7 kg/85.0%, purity 99.9%). The crude product (18.4 kg) was dissolved into isopropanol (144.5 kg) and filtrated hot. The filtrate was concentrated at atmospheric pressure by distillation off isopropanol about 74 kg. The residue was cooled slowly and the solid was collected by filtration at about 5° C., washed with water and isopropanol and finally dried under reduced pressure to afford 6-(3,5-dimethylisoxazol-4-yl)-7-methoxy-3-methyl-1-(pyridin-2-ylmethyl)quinolin-2(1H)-one (17.17 kg/93.3%, purity 100%). The product was crystalline form 1 of compound (I).

### Example 6. Alternative method for the preparation of 6-(3, 5-Dimethylisoxazol-4-yl)-7-methoxy-3-methyl-1-(pyridin-2-ylmethyl)quinolin-2(1H)-one (I) crystalline form 1

n-Butanol (581 g), water (175 g), 6-bromo-7-methoxy-3-methyl-1-(pyridin-2-ylmethyl)quinolin-2(1H)-one (175 g), potassium carbonate (135 g), 3,5-dimethyl-4-(4,4,5,5-tetramethyl-1,3,2-dioxaborolan-2-yl)isoxazole (164 g) and tetrakis(triphenylphosphine)palladium (5.24 g) were charged to a reactor under nitrogen atmosphere. The mixture was heated to boiling for 3-4 hours. When the reaction was complete, the reaction mixture was concentrated by distilling off about 710 ml of the solvents. To the distillation residue, toluene (727 g) and water (525 g) were added. The phases were separated at elevated temperature. The organic phase was filtered through celite. Toluene (280 g) was used to flush the filter. Combined filtrates were concentrated by distilling off about 940 ml of the solvents. The residue was cooled and the solid was collected by filtration at about 5° C., washed with water and isopropanol and finally dried under reduced pressure to yield the crude title compound (145.8 g/79.7%, purity 99.7%). 50 g of the crude product was dissolved into isopropanol (392 g) and filtrated hot. The filtrate was concentrated at atmospheric pressure by distillation off isopropanol about 155 nil. The residue was cooled and the solid was collected by filtration at about 5° C., washed with isopropanol and finally dried under reduced pressure to afford 6-(3,5-dimethylisoxazol-4-yl)-7-methoxy-3-methyl-1-(pyridin-2-ylmethyl)quinolin-2(1H)-one (47.4 g/94.8%, purity 100.0%). The product was crystalline form 1 of compound (I).

### Example 7. Alternative method for the preparation of 6-(3, 5-Dimethylisoxazol-4-yl)-7-methoxy-3-methyl-1-(pyridin-2-ylmethyl)quinolin-2(1H)-one (I) crystalline form 1

n-Butanol (32.4 g), water (10 g), 6-bromo-7-methoxy-3-methyl-1-(pyridin-2-ylmethyl)quinolin-2(1H)-one (10 g), triphenylphosphine (0.19 g) potassium carbonate (7.69 g), 3,5-dimethyl-4-(4,4,5,5-tetramethyl-1,3,2-dioxaborolan-2-yl)isoxazole (8.38 g) and palladium acetate (0.047 g) were charged to a reactor. The mixture was heated to boiling for about 6 hours. When the reaction was complete, the water phase was separated. The above reaction was repeated using another batch of starting material 6-bromo-7-methoxy-3-methyl-1-(pyridin-2-ylmethyl)quinolin-2(1H)-one (10 g). The organic phases from the first batch and the repeated batch were combined. Toluene (72.7 g) was added to the combined organic phases and the solution was filtered through celite at elevated temperature. Toluene (28 g) was used to flush the filter. Combined filtrates were concentrated by distilling off about 130 ml of the solvents. The residue was cooled and the solid was collected by filtration at about 5° C., washed with water and isopropanol and finally dried under reduced pressure to yield 6-(3,5-dimethylisoxazol-4-yl)-7-methoxy-3-methyl-1-(pyridin-2-ylmethyl)quinolin-2(1H)-one (17.3 g/82.6%, purity 99.9%). The product was crystalline form 1 of compound (I).

### Example 8. Preparation of amorphous 6-(3, 5-Dimethylisoxazol-4-yl)-7-methoxy-3-methyl-1-(pyridin-2-ylmethyl)quinolin-2(1H)-one (I)

1 g of 6-(3, 5-Dimethylisoxazol-4-yl)-7-methoxy-3-methyl-1-(pyridin-2-ylmethyl)quinolin-2(1H)-one (I) crystalline form 1 was melted at 180° C. under protective nitrogen flow and cooled to room temperature with natural cooling rate. The obtained material was found to be amorphous by XRPD analysis (FIG. 4).

### Example 9. Preparation of crystalline form 2 of 6-(3, 5-Dimethylisoxazol-4-yl)-7-methoxy-3-methyl-1-(pyridin-2-ylmethyl)quinolin-2(1H)-one (I)

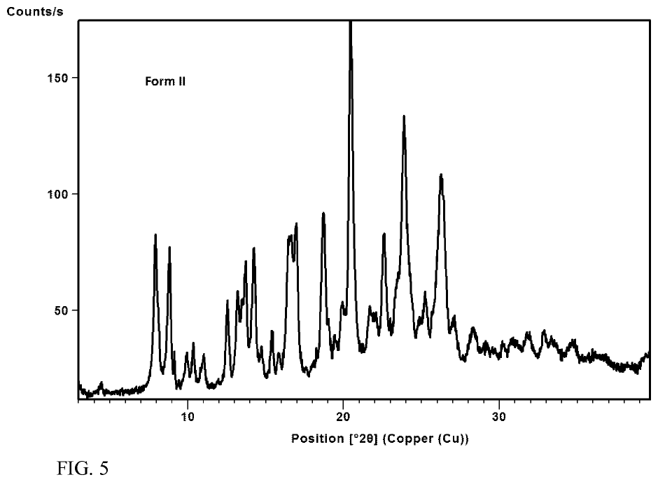

Amorphous 6-(3,5-dimethylisoxazol-4-yl)-7-methoxy-3-methyl-1-(pyridin-2-ylmethyl)quinolin-2(1H)-one (I) was milled in mortar and placed in the 10 ml glass bottle and 5 ml of water was added. The slurry was left under the hood for 8 weeks. The obtained solid material was isolated by filtering, air-dried and forwarded to XRPD analysis. The product was found to be crystalline form 2 of compound (I) (FIG. 5).

### Example 10. Alternative method for the preparation of 6-(3, 5-Dimethylisoxazol-4-yl)-7-methoxy-3-methyl-1-(pyridin-2-ylmethyl)quinolin-2(1H)-one (I) crystalline form 2

20 mg of 6-(3,5-dimethylisoxazol-4-yl)-7-methoxy-3-methyl-1-(pyridin-2-ylmethyl)quinolin-2(1H)-one (I) was dissolved in 3 ml of 2-propanol. The solution was added fast to 12 ml of water which was pre-cooled to 4° C. The mixture was aged for 24 h at 4° C. The solids were filtered and air-dried. The obtained solid material was isolated by filtering, air-dried and forwarded to XRPD analysis. The product was found to be crystalline form 2 of compound (I) with traces of form 1.

### Example 11. XRPD studies of various forms of 6-(3,5-dimethylisoxazol-4-yl)-7-methoxy-3-methyl-1-(pyridin-2-ylmethyl)quinolin-2(1H)-one (I)

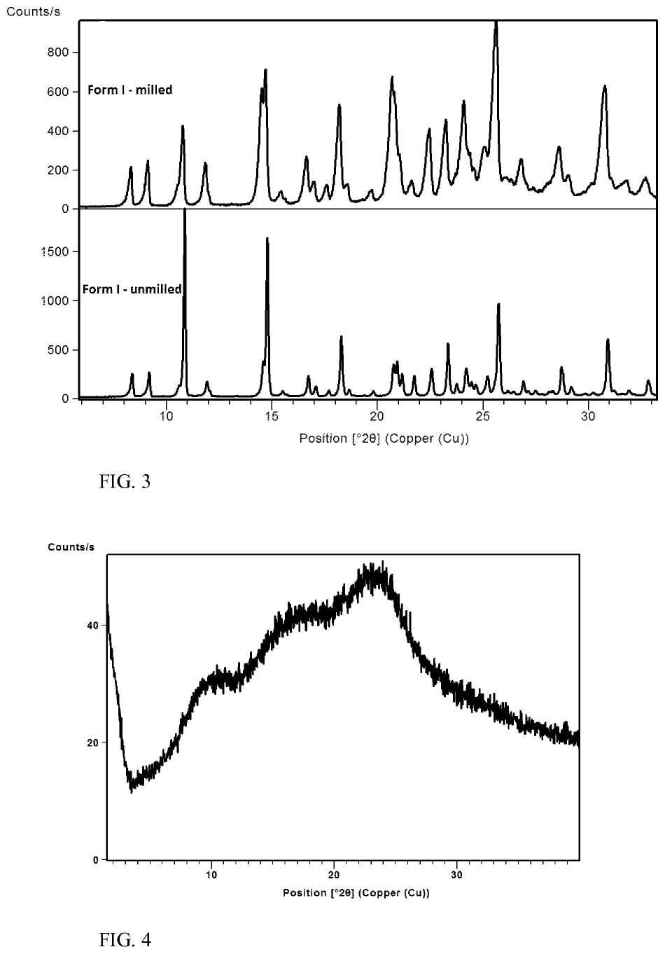

The XRPD pattern of the crystalline form 1 of compound (I) was measured using a PANalytical X'Celerator θ-θ diffractometer with CuKα radiation (40 kV, 30 mA). The diffractometer was operated in reflection mode. The measurements were performed in the range of 3°-40° 2θ. 100-300 mg of the sample powder was placed in the sample holder and the surface was pressed. The XRPD pattern of an unmilled sample of crystalline form 1 is shown in FIG. 1. The XRPD pattern of a milled sample of crystalline form 1 is shown in FIG. 2. For comparison purposes, the XRPD pattern of an unmilled sample of crystalline form 1 and the XRPD pattern of a milled sample of crystalline form 1 are shown together in FIG. 3.

The XRPD pattern of the amorphic form and the crystalline form 2 of compound (I) were measured as above except that a small sample amounts (approximately 5-10 mg) were first placed in the centre of a zero-background sample holder and then gently spread to a thin layer. The XRPD pattern of the amorphic form of compound (I) prepared according to Example 8 is shown in FIG. 4. The XRPD pattern of a milled sample of crystalline form 2 prepared according to Example 9 is shown in FIG. 5.

The XRPD pattern of crystalline form 2 of compound (I) comprises the characteristic peaks at about 7.9, 8.8, 13.2, 13.7 and 14.2 degrees 2-theta, particularly at about 4.4, 7.9, 8.8, 12.5, 13.2, 13.7 and 14.2 degrees 2-theta, still more particularly at about 4.4, 7.9, 8.8, 12.5, 13.2, 13.7, 14.2, 20.4 and 26.2 degrees 2-theta.

### Example 12. Stability Studies

Four samples of compound (I) in crystalline form 2 (with traces of form 1) prepared according to Example 10 were stored at stressed conditions. XRPD of each sample was recorded at initial time point and at further time points. The results and the conditions used are shown in the Table 1. Moreover, long term stability of crystalline form 1 of compound (I) at different storage conditions was studied. The results are shown in Table 2.

TABLE 1
Relative stability study between crystalline form 1 and 2

| | Time point | | | | | | |
|---|---|---|---|---|---|---|---|
| Condition | | 2 | 4 | 12 | 24 | 36 | 48 |
| ° C./RH % | initial | weeks | weeks | weeks | weeks | weeks | weeks |
| 25/60 | II + | II + | II + | II + | I + | I | I |
| | I(traces) | I(traces) | I(traces) | I(traces) | I(traces) | | |
| 40/75 | II + | II + | II + | I | n.d. | n.d. | I |
| | I(traces) | I(traces) | I(traces) | | | | |
| 30/65 | II + | II + | II + | II + | I + | I | I |
| | I(traces) | I(traces) | I(traces) | I(traces) | I(traces) | | |
| RT/5 | II + | I | n.d. | n.d. | n.d. | n.d. | I |
| | I(traces) | | | | | | |

RT = room temperature
n.d. = not determined

It can be seen that form 1 is more stable than form 2 since form 2 transformed to form 1 within 36 weeks at all studied conditions.

TABLE 2
Long-term stability of crystalline form 1
at different storage conditions

| Storage condition | Crystalline form 1 |
|---|---|
| 25° C./RH 60 % | Stable up to 24 months |
| 40° C./RH 75 % | Stable up to 6 months |

It can be seen that crystalline form 1 of compound (I) exhibits a good long-term stability as no changes were observed by XRPD.

1. Crystalline form 1 of 6-(3,5-dimethylisoxazol-4-yl)-7-methoxy-3-methyl-1-(pyridin-2-ylmethyl)quinolin-2(1H)-one (I) having an X-ray powder diffraction pattern comprising characteristic peaks at about 10.8, 25.6, and 30.7 degrees 2-theta.

2. The crystalline form 1 of 6-(3,5-dimethylisoxazol-4-yl)-7-methoxy-3-methyl-1-(pyridin-2-ylmethyl)quinolin-2(1H)-one (I) according to claim 1, further having an X-ray powder diffraction pattern comprising a peak at about 14.7 degrees 2-theta.

3. The crystalline form 1 of 6-(3,5-dimethylisoxazol-4-yl)-7-methoxy-3-methyl-1-(pyridin-2-ylmethyl)quinolin-2(1H)-one (I) according to claim 2, further having an X-ray powder diffraction pattern comprising peaks at about 8.3, 11.8 and 18.2 degrees 2-theta.

4. (canceled)

5. The crystalline form 1 of 6-(3,5-dimethylisoxazol-4-yl)-7-methoxy-3-methyl-1-(pyridin-2-ylmethyl)quinolin-2(1H)-one (I) according to claim 1, wherein the crystalline form 1 of 6-(3, 5-dimethylisoxazol-4-yl)-7-methoxy-3-methyl-1-(pyridin-2-ylmethyl)quinolin-2(1H)-one (I) is an anhydrate.

6. A method of preparing the crystalline form 1 of 6-(3, 5-dimethylisoxazol-4-yl)-7-methoxy-3-methyl-1-(pyridin-2-ylmethyl)quinolin-2(1H)-one (I) according to claim 1, comprising:
a) reacting 6-bromo-7-methoxy-3-methyl-1-(pyridin-2-ylmethyl)quinolin-2(1H)-one of formula (Ia) 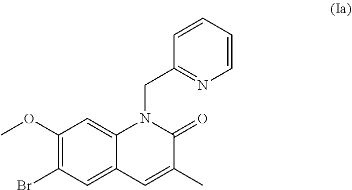 [SMILES: COc1cc2c(cc1Br)cc(C)c(=O)n2Cc1ccccn1]
with a boronic acid derivative of formula (Ib) or (Ic) 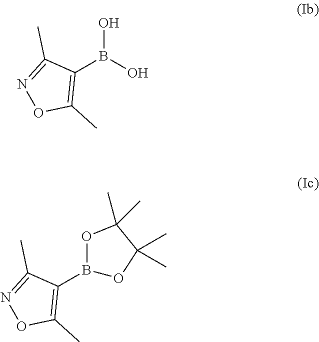
at an elevated temperature in the presence of a palladium catalyst and a base in an acetonitrile-water or n-butanol-water solvent;
b) optionally isolating the organic phase of the reaction mixture;
c) adding toluene and optionally water to the reaction mixture or to the organic phase of the reaction mixture if it was isolated in step b);
d) isolating the organic phase if water was added in step c);
e) concentrating the organic phase by distillation; and
f) cooling the concentrated organic phase and isolating the precipitated crystalline form 1 of 6-(3,5-dimethylisoxazol-4-yl)-7-methoxy-3-methyl-1-(pyridin-2-ylmethyl)quinolin-2(1H)-one (I).

7. The method according to claim 6, wherein the amount of palladium catalyst used per amount of 6-bromo-7-methoxy-3-methyl-1-(pyridin-2-ylmethyl)quinolin-2(1H)-one of formula (Ia) in step a) is from about 0.3 to about 2 mol %.

8. The method according to claim 7, wherein the palladium catalyst is Pd(PPh₃)₄ or a combination of Pd(OAc)₂ and triphenylphosphine.

9. The method according to claim 8, wherein the molar ratio of Pd(OAc)₂ to triphenylphosphine is 1:3.

10. The method according to claim 6, wherein the base is sodium carbonate or potassium carbonate.

11. The method according to claim 6, wherein after step f) the crystalline form 1 of 6-(3,5-dimethyl-isoxazol-4-yl)-7-methoxy-3-methyl-1-(pyridin-2-ylmethyl)quinolin-2(1H)-one (I) is recrystallized from isopropanol.

12. The method according to claim 6, wherein the organic phase is filtered through celite before step e).

13. The method according to claim 6, wherein the acetonitrile-water solvent and the boronic acid derivative of formula (Ib) are used in step a).

14. The method according to claim 6, wherein the n-butanol-water solvent and the boronic acid derivative of formula (Ic) are used in step a).

15. The method according to claim 6 comprising:
a) reacting 6-bromo-7-methoxy-3-methyl-1-(pyridin-2-ylmethyl)quinolin-2(1H)-one of formula (Ia) 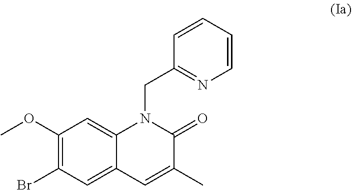 [SMILES: COc1cc2c(cc1Br)cc(C)c(=O)n2Cc1ccccn1]
with a boronic acid derivative of formula (Ib) 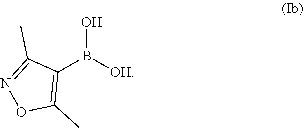
at an elevated temperature in the presence of a palladium catalyst and a base in an acetonitrile-water solvent;
b) optionally concentrating the reaction mixture by distillation;
c) adding toluene and water to the reaction mixture;
d) isolating the organic phase;
e) concentrating the organic phase by distillation; and
f) cooling the concentrated organic phase and isolating the precipitated crystalline form 1 of 6-(3,5-dimethylisoxazol-4-yl)-7-methoxy-3-methyl-1-(pyridin-2-ylmethyl)quinolin-2(1H)-one (I).

16. The method according to claim 15, wherein the amount of palladium catalyst used per amount of 6-bromo-7-methoxy-3-methyl-1-(pyridin-2-ylmethyl)quinolin-2(1H)-one of formula (Ia) in step a) is from about 0.3 to about 2 mol %.

17. The method according to claim 16, wherein the palladium catalyst is Pd(PPh₃)₄.

18. The method according to claim 15, wherein the base is potassium carbonate.

19. The method according to claim 15, wherein the organic phase is filtered through celite before step e).

20. The method according to claim 15, wherein after step f) the crystalline form 1 of 6-(3,5-dimethyl-isoxazol-4-yl)-7-methoxy-3-methyl-1-(pyridin-2-ylmethyl)quinolin-2(1H)-one (I) is recrystallized from isopropanol.

21. The method according to claim 6 comprising:
a) reacting 6-bromo-7-methoxy-3-methyl-1-(pyridin-2-ylmethyl)quinolin-2(1H)-one of formula (Ia) 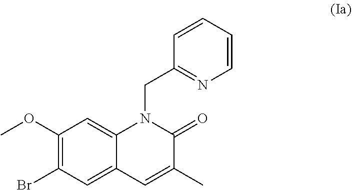 [SMILES: COc1cc2c(cc1Br)cc(C)c(=O)n2Cc1ccccn1]
with a boronic acid derivative of formula (Ic) 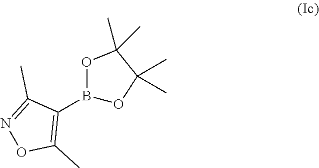 [SMILES: Cc1noc(C)c1B1OC(C)(C)C(C)(C)O1]
at an elevated temperature in the presence of a palladium catalyst and a base in a n-butanol-water solvent;
b) optionally concentrating the reaction mixture by distillation;
c) adding toluene and water to the reaction mixture;
d) isolating the organic phase;
e) concentrating the organic phase by distillation; and
f) cooling the concentrated organic phase and isolating the precipitated crystalline form 1 of 6-(3,5-dimethylisoxazol-4-yl)-7-methoxy-3-methyl-1-(pyridin-2-ylmethyl)quinolin-2(1H)-one (I).

22. The method according to claim 21, wherein the amount of palladium catalyst used per amount of 6-bromo-7-methoxy-3-methyl-1-(pyridin-2-ylmethyl)quinolin-2(1H)-one of formula (Ia) in step a) is from about 0.3 to about 2 mol %.

23. The method according to claim 22, wherein the palladium catalyst is Pd(PPh₃)₄.

24. The method according to claim 21, wherein the base is potassium carbonate.

25. The method according to claim 21, wherein the organic phase is filtered through celite before step e).

26. The method according to claim 21, wherein after step f) the crystalline form 1 of 6-(3,5-dimethyl-isoxazol-4-yl)-7-methoxy-3-methyl-1-(pyridin-2-ylmethyl)quinolin-2(1H)-one (I) is recrystallized from isopropanol.

27. The method according to claim 6 comprising:
a) reacting 6-bromo-7-methoxy-3-methyl-1-(pyridin-2-ylmethyl)quinolin-2(1H)-one of formula (Ia) 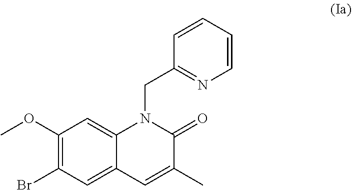 [SMILES: COc1cc2c(cc1Br)cc(C)c(=O)n2Cc1ccccn1]
with a boronic acid derivative of formula (Ic) 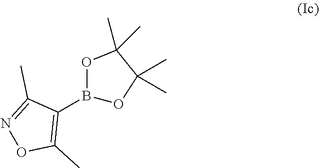 [SMILES: Cc1noc(C)c1B1OC(C)(C)C(C)(C)O1]
at an elevated temperature in the presence of a palladium catalyst and a base in a n-butanol-water solvent;
b) isolating the organic phase of the reaction mixture;
c) adding toluene to the organic phase;
d) concentrating the organic phase by distillation; and
e) cooling the concentrated organic phase and isolating the precipitated crystalline form 1 of 6-(3,5-dimethylisoxazol-4-yl)-7-methoxy-3-methyl-1-(pyridin-2-ylmethyl)quinolin-2(1H)-one (I).

28. The method according to claim 27, wherein the amount of palladium catalyst used per amount of 6-bromo-7-methoxy-3-methyl-1-(pyridin-2-ylmethyl)quinolin-2(1H)-one of formula (Ia) in step a) is from about 0.3 to about 2 mol %.

29. The method according to claim 28, wherein the palladium catalyst is a combination of Pd(OAc)₂ and triphenylphosphine.

30. The method according to claim 29, wherein the molar ratio of Pd(OAc)₂ to triphenylphosphine is 1:3.

31. The method according to claim 27, wherein the base is potassium carbonate.

32. The method according to claim 27, wherein the organic phase is filtered through celite before step d).

33. The method according to claim 27, wherein after step e) the crystalline form 1 of 6-(3,5-dimethyl-isoxazol-4-yl)-7-methoxy-3-methyl-1-(pyridin-2-ylmethyl)quinolin-2(1H)-one (I) is recrystallized from isopropanol.

34. The method according to claim 6, wherein the 6-bromo-7-methoxy-3-methyl-1-(pyridin-2-ylmethyl)quinolin-2(1H)-one of formula (Ia) is prepared by a method comprising:
(i) reacting 4-bromo-methoxyaniline with propionic anhydride in a solvent to obtain a compound of formula (IV); 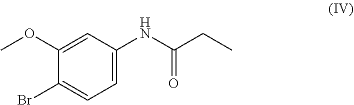
(ii) treating the compound of formula (IV) with phosphorous oxychloride and dimethyl formamide in a solvent to obtain a compound of formula (III); 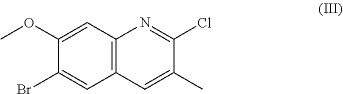
(iii) treating the compound of formula (III) with acetic acid and water at elevated temperature to obtain a compound of formula (II); and 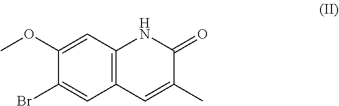
(iv) reacting the compound of formula (II) with 2-(chloromethyl)pyridine or a salt thereof in a solvent at elevated temperature in the presence of a base followed by isolation of the 6-bromo-7-methoxy-3-methyl-1-(pyridin-2-ylmethyl)-quinolin-2(1H)-one of formula (Ia).
